(19) United States
(12) Patent Application Publication
(10) Pub. No.: US 2010/0120072 A1
(43) Pub. Date: May 13, 2010

(54) DETECTION OF ELEVATED LEVELS OF HER-2/NEU PROTEIN FROM NON-ISOLATED CIRCULATING CANCER CELLS AND TREATMENT

(75) Inventors: Robert M. Lorence, Bethesda, MD (US); Ming Lu, Potomac, MD (US)

Correspondence Address: Wellstat Management Company. LLC, LEGAL DEPARTMENT, 930 CLOPPER ROAD, GAITHERSBURG, MD, 20878, US

(73) Assignee: WELLSTAT BIOLOGICS CORPORATION, Gaithersburg, MD (US)

(21) Appl. No.: 12/593,160

(22) PCT Filed: Apr. 15, 2008
(86) PCT No.: PCT/US2008/060317

§ 371 Date: Sep. 25, 2009

Related U.S. Application Data
(60) Provisional application No. 60/912,833, filed on Apr. 19, 2007.

(51) Int. Cl. G01N 33/574

(52) U.S. Cl. 435/7.23

(57) ABSTRACT

The expression of Her-2/neu protein on circulating cancer cells in a sample of blood or peripheral blood mononuclear cells (PBMCs) is detected by performing a sensitive Her-2/neu immunoassay. There is no need to isolate the cancer cells before performing the immunoassay. A positive result indicates the expression of Her-2/neu on cancer cells in the blood sample. This method can be used to identify cancer patients who are likely to benefit from treatment with an anticancer agent that targets Her-2/neu, such as trastuzumab (HERCEPTIN), lapatinib, CP-724,714, NKI-272, and BMS-599626

### BACKGROUND OF THE INVENTION

Current tests on the market to detect Her-2/neu (also called Her2/neu; HER2; c-erbB-2 and erbB2) protein or associated gene from cancer cells are cumbersome and very time consuming. Furthermore, only 25 to 30% of breast cancer patients receive Her-2/neu directed therapy based on the findings of elevated levels of the Her-2/neu (also called Her2/neu; HER2; c-erbB-2 and erbB2) protein or gene in biopsies of their primary tumor. The data in the literature suggest that a significant number of women (11 of 26 tested) with negative results for Her2/neu in their primary tumor biopsy go on to develop Her2/neu positivity on their circulating cancer cells (Hayes D F, et al., Int J Oncol 21:1111-7; Meng S et al., 2004 Proc Natl Acad Sci USA 101:9393-98). Also, a considerable number of women with breast cancer do not have biopsy material readily available for testing for Her-2-neu status. The approaches used in the papers by Hayes et al and by Meng et al are cumbersome and time-consuming and a rapid more convenient test is needed, especially one that can be done in the physician's office. The method of Hayes et al. requires flow cytometric analysis and the method of Meng et al. requires fluorescence in situ hybridization (FISH) which are more complicated and time-consuming than direct detection (e.g., by ECL) of Her-2/neu protein as noted in this invention. Furthermore, the methods of Meng et al. and Hayes et al. required the immunomagnetic beads to enrich the tumor cells. In constast, a rapid method as in this invention that can be performed without the need for such isolation steps offers further advantages in terms of improved sensitivity by avoiding losses seen with steps used to isolate cancer cells from either whole blood or peripheral blood mononuclear cells.

Her2/neu and Her-2/neu targeted treatment: Overexpression of Her2/neu oncogene is observed in approximately 25% of biopsy samples from women with breast cancer and is associated with a poor prognosis. Trastuzumab (HERCEPTIN) is a humanized monoclonal antibody that is directed against the extracellular domain (ECD) of the Her2/neu receptor and inhibits the proliferation of human breast cancer cells overexpressing this receptor (see Esteve F J 2004, The Oncologist 9(Suppl 3):pp 4-9 for a recent review). Protein expression of Her-2/neu on breast cancer cells can easily reach levels of 500,000 molecules or more per cell as is the case of the Her-2/neu overexpressing human breast cancer cell line called SK-BR-3. Current tests for Her2/neu rely on testing tissue sections of the patients' biopsy for overexpression of the protein or for gene amplification. In those women with gene amplification or at least 2+ immunostaining for the protein, significant responses have been demonstrated with single agent trastuzumab or with trastuzumab in combination with a chemotherapeutic such as paclitaxel. Recently, lapatinib (also called GW-572016) was approved by the U.S. Food and Drug Administration (FDA) for the treatment of women with Her-2/neu positive breast cancer. Besides breast cancer, Her-2/neu is overexpressed on other carcinoma cells including ovarian carcinoma.

There is a significant need in medical practice for a convenient assay that can be performed rapidly and directly from a whole blood or PBMC sample that is sensitive and specific enough to identify those women with breast cancer who have overexpression of Her-2/neu protein on circulating breast cancer cells and who therefore are likely to benefit from Her-2/neu-targeting therapy, e.g. trastuzumab, lapatinib, or other anti-Her-2/neu-directed therapies. This is all the more important since there is evidence in the literature indicate that thousands of women with breast cancer, who are not currently eligible for trastuzumab or lapatinib treatment, have circulating tumor cells with overexpression of Her-2/neu and who have primary tumors that were deemed negative for Her-2/neu. Such women could benefit from anti-Her-2/neu targeted agents if they are first identified.

A method for detecting the expression of Her-2/neu protein on isolated circulating cancer cells is described in WO 2006/041959 (Wellstat Biologics Corp.). Eliminating the need to first isolate the circulating cancer cells would be a significant advantage in terms of ease of testing, rapidity of the assay, and, most importantly, the prevention of loss of cancer cells by reducing extensive manipulation of the sample. But the art considered there to be major obstacles in obtaining an immunoassay with sufficient sensitivity and specificity in order to detect Her-2/neu from low levels of circulating breast cancer cells in a whole blood sample or from a sample of human peripheral blood mononuclear cells.

Both Hayes et al. (2002, Int J Oncol 21:1111-7) and Meng et al. (2004 Proc Natl Acad Sci USA 101:9393-98) required use of enrichment in their assays for Her-2/neu from circulating cancer cells. Using flow cytometry to detect Her-2/neu on circulating cancer cells, Hayes et al. indicates that such enrichment is “required” and “essential” in order to achieve the sensitivity needed (see page 1112 last sentence and page 1113 first sentence). In addition, Leone et al. (2003; J Leukocyte Biol 74:593-601) indicate that both Her-2/neu transcript and protein are expressed at low levels in normal peripheral blood mononuclear cells (PMBCs). Such Her-2/neu expression indicated by Leone et al. would be expected to make it difficult to look at Her-2/neu expression in circulating cancer cells in the presence of high numbers of PBMCs.

### SUMMARY OF THE INVENTION

This invention provides a method of detecting the expression of Her-2/neu protein on circulating cancer cells in a whole blood sample, comprising performing on the blood sample an immunoassay capable of detecting cancer cell-associated Her-2/neu, in which a positive immunoassay result indicates the presence of Her-2/neu on the cancer cells; wherein the circulating cancer cells are not isolated from the whole blood prior to the performance of the immunoassay; and wherein the immunoassay: a) is capable of detecting Her-2/neu from SK-BR-3 breast cancer cells when spiked into blood at a concentration of less than or equal to 100 SK-BR-3 cells per milliliter of blood; and b) is capable of detecting Her-2/neu from 10 SK-BR-3 breast cancer cells when assayed in the presence of at least 1 million human peripheral blood mononuclear cells.

This invention provides a method of detecting the expression of Her-2/neu protein on circulating cancer cells in a blood sample, comprising performing on the blood sample an immunoassay capable of detecting cancer cell-associated Her-2/neu, in which a positive immunoassay result indicates the presence of Her-2/neu on the cancer cells; wherein the circulating cancer cells are not isolated from peripheral blood mononuclear cells prior to the performance of the immunoassay; and wherein the immunoassay: a) is capable of detecting Her-2/neu from SK-BR-3 breast cancer cells when spiked into blood at a concentration of less than or equal to 100 SK-BR-3 cells per milliliter of blood; and is capable of detecting Her-2/neu from 10 SK-BR-3 breast cancer cells when assayed in the presence of at least 1 million human peripheral blood mononuclear cells.

This invention is based on the finding that circulating cancer cells do not need to be isolated in order to detect the expression of Her-2/neu protein on such cells. Eliminating the need to first isolate the circulating cancer cells is a significant advantage in terms of ease of testing, rapidity of the assay, and, most importantly, the prevention of loss of cancer cells by reducing extensive manipulation of the sample. Having fewer steps makes the method of this invention quicker and less expensive to perform. Also, having fewer steps makes the method easier to automate. Furthermore, the improved assay of the invention prevents loss of cancer cells by eliminating isolation steps, which can have a significant negative effect on the sensitivity of detecting Her-2/neu from circulating cancer cells in a blood sample.

This invention provides a method of identifying a cancer patient likely to benefit from treatment with an anticancer agent that targets Her-2/neu, comprising the detection methods described above. When used to identify such patients, the cancer cell-containing blood sample is drawn from the patient. This invention provides a method of treating cancer patients so identified, which method comprises administering a Her-2/neu-targeting anticancer agent to the patient.

### BRIEF DESCRIPTION OF THE FIGURES

FIG. 1. Comparison of the ECL signal for the immunoassay detection of Her-2/neu in lysates from SK-BR-3 breast cancer cells (positive control for Her-2/neu overexpression) versus MDA-MB-468 (negative control for Her-2/neu overexpression). Shown is the data using lysates from 1, 2, and 10 cells per well.

FIG. 2. Further comparison of the ECL signal for the immunoassay detection of Her-2/neu in lysates from SK-BR-3 breast cancer cells (positive control for Her-2/neu overexpression) versus MDA-MB-468 (negative control for Her-2/neu overexpression). Data from the same experiment as shown in FIG. 1 is used for this figure, except that data obtained from lysate material from 50 and 250 cells per well were also included in this graph in order to display an increased X-axis scale.

### DETAILED DESCRIPTION OF THE INVENTION

As used herein the transitional term “comprising” is open-ended. A claim utilizing this term can contain elements in addition to those recited in such claim. Thus, for example, the claims can read on methods that also include other steps not specifically recited therein, as long as the recited elements or their equivalent are present.

The terms “non-isolated cancer cells” or “cancer cells not isolated”: As used herein, the terms “non-isolated cancer cells” or “cancer cells not isolated” in reference to cancer cells with antigens for assay testing refer to cancer cells being tested for antigen in the presence of at least 100,000 non-cancer cells per ml of sample, more preferably in the presence of at least 500,000 non-cancer cells per ml of sample, more preferably in the presence of at least 1 million non-cancer cells per ml of sample, and most preferably in the presence of at least 2 million non-cancer cells per ml of sample. For example, if an assay is focused on an antigen found on circulating human cancer cells, and if circulating human cancer cells are in the presence of 500,000 human peripheral blood mononuclear cells per ml in an assay sample, then the circulating human cancer cells in the sample are cancer cells that are not isolated and are non-isolated cancer cells.

In one embodiment of this invention the immunoassay is a solution-based immunoassay. Solution-based immunoassay: As used herein, the term “solution-based immunoassay” refers to an immunoassay of an antigen in solution using at least one antibody against the antigen. Detection techniques suitable for solution-based immunoassays include electrochemiluminescence, chemiluminescence, fluorogenic chemiluminescence, fluorescence polarization, and time-resolved fluorescencen.

In another embodiment of this invention the immunoassay is a sandwich immunoassay. As used herein, the term “sandwich immunoassay” refers to an assay to detect antigen using a pair of antibodies (for example, antibody ‘A’ and antibody ‘B’) each directed against the antigen or a portion of the antigen. For the pair of antibodies, antibody ‘A’ is labeled either covalently or non-covalently to a reporter molecule (e.g., a molecule that allows for electrochemiluminescence or a molecule that allows for fluorescence). An example of non-covalent labeling of an antibody ‘A’ would be to allow a secondary labeled antibody against the antibody ‘A’ to bind to antibody ‘A’. Antibody ‘B’ is attached directly (or allowed to attach indirectly) to a solid support phase like an assay plate, a magnet or an electrode. Detection techniques suitable for sandwich immunoassays include electrochemiluminescence, chemiluminescence, and fluorogenic chemiluminescence.

This invention provides methods sensitive enough for quantifying the levels of Her-2/neu protein on circulating breast cancer cells in blood samples and provides methods for identifying those women with breast cancer who are likely to benefit from therapy using trastuzumab, lapatinib, or another agent targeted to Her-2/neu. Other agents which can target Her-2/neu are in development and have a similar applicability as trastuzumab or lapatinib for this invention. These include but are not limited to: OMNITARG (pertuzumab) being developed by Genentech; CP-724,714 and CP-654577 being developed by Pfizer (Munster et al., 2007, Clin Cancer Res 13:1238-45; Barbacci et al., 2003, Cancer Res 63:4450-4459); HKI-272 being developed by Wyeth (Wong et al., 2006, J Clin Oncol 24(June 20 Supplement):3018; Rabindran et al., 2004, Cancer Res 2004, 64:3958-65); and BMS-599626 being developed by Bristol-Myers-Squibb. Other anticancer agents that include Her-2/neu among their specificity are described in Spector et al., 2007 (Breast Cancer Res 9, beginning on page 205) and Janmaat and Giaccone, 2003 (The Oncologist 8:576-86). Additional anticancer agents that target Her-2/neu also include Her2 targeted nanoparticle bioconjugates (see Alexis F, et al., 2007; Abstract #4181, 2007 American Association for Cancer Research Annual Meeting) and anti-Her2 immunoliposomes containing chemotherapeutic agents (see Noble CO et al., 2004, Expert Opin Ther Targets 8:335-53).

The immunoassay utilized in accordance with this invention is sensitive enough to detect Her-2/neu from at least 100 SK-BR-3 breast cancer cells spiked per milliliter of blood, preferably sensitive enough to detect at least 30 SK-BR-3 breast cancer cells spiked per milliliter of blood, more preferably sensitive enough to detect at least 10 SK-BR-3 breast cancer cells spiked per milliliter of blood, more preferably sensitive enough to detect at least 3 SK-BR-3 breast cancer cells spiked per milliliter of blood, and most preferably sensitive enough to detect at least 1 SK-BR-3 breast cancer cells spiked per milliliter of blood. The immunoassay utilized in accordance with this invention is resistant to interference such that it is capable of detecting Her-2/neu from 10 SK-BR-3 breast cancer cells when assayed in the presence of at least 1 million human peripheral blood mononuclear cells.

In a preferred embodiment, the immunoassay generates a signal proportional to the number of cancer cell-associated Her-2/neu molecules in the blood sample.

A sample (usually in the range of approximately 8 to 20 ml) of blood from a patient with cancer, especially breast cancer, is taken. Steps include as detailed below:
1. Optional depletion of erythrocytes and neutrophils. The preferred embodiment includes this step.
2. Detection and quantification of the Her-2/neu protein from circulating carcinoma cells

1. Optional depletion of erythrocytes and neutrophils. A preferred method uses the BD Vacutainer CPT tubes with anticoagulant (EDTA or citrate). These tubes contain a material that upon correct centrifugation (1,100×g for 10 minutes, swing-out bucket rotor) allows for elimination of red blood cells and neutrophils. After centrifugation, the bottom of the tube contains a cell pellet of erythrocytes (red blood cells) and neutrophils. Above the cell pellet is a gel barrier and above the gel barrier are the mononuclear cells (tumor cells, lymphocytes and monocytes) as a band at the bottom of the plasma. The tumor cells, lymphocytes and monocytes can then be readily collected from the top above the gel barrier. This method is preferred as it removes not only the red blood cells but also the neutrophils.

2. Detection and quantification of the Her-2/neu protein from circulating carcinoma cells.

Detection of Her-2/neu can then be accomplished by use of a monoclonal antibody (mAb) such as HERCEPTIN or a polyclonal antibody against Her-2/neu (e.g., Goat polyclonal antibody catalog number AF1129 from R&D systems) that are linked either directly or indirectly to a detecting molecule. In the case of electrochemiluminescence (ECL), the detecting molecule is ruthenium. There is abundant literature in the public domain provides amply useful methods for linking ruthenium to antibodies (eg, Lee et al., Am J Trop Med Hyg 2001, 65:1-9) followed by ECL detection of antigens on magnetic beads in a solution containing tripropylamine. With application of an electric potential, the ruthenium label is excited and light is emitted and detected using an ECL detecting instrument (such as the ORIGEN analyzer or a commercially available instrument like the M-Series® 384 from BIOVERIS Corporation, Gaithersburg, Md.)

The immunoassay utilized in accordance with this invention consists of at least one antibody, and preferably two sets of antibodies. These antibodies can be either a polyclonal or a monoclonal antibody against Her-2/neu. Preferably the monoclonal antibody is a humanized mouse monoclonal antibody, e.g. trastuzumab. Trastuzumab is a preferred embodiment for the immunoassay and treatment methods in accordance with this invention.

In an additional embodiment of the invention, a secondary antibody against one of the antibodies targeting Her-2/neu is used. In a preferred embodiment of the invention, the secondary antibody is labeled either covalently or non-covalently to a reporter molecule (e.g., a molecule that allows for electrochemiluminescence or a molecule that allows for fluorescence). In another preferred embodiment of the invention, ECL is used and the secondary antibody is biotinylated which allows for attachment of the secondary antibody to a streptavidin-coated magnetic bead.

For purposes of detection of Her-2/neu, a variety of monoclonal and polyclonal antibodies against Her-2/neu and include antibodies against the extracellular domain and against the cytoplasmic domain are commercially available from such sources as R&D Systems (Minneapolis, Minn. Biosource (Camarillo, Calif.) and BD Biosciences, San Diego, Calif.). Rabbit polyclonal antibodies are also available from LABVISION Corp, Fremont. CA; such as neu Ab-21) and from UPSTATE CELL SIGNALING SOLUTIONS (Lake Placid, N.Y.; such as Catolog number 06-562). A goat polyclonal antibody against the extracellular domain from Her-2/neu is available from R&D systems (catalog number AF1129). A goat polyclonal antibody against full-length recombinant Her-2/neu is available from EXALPHA BIOLOGICS (Rosedale, Mass.; catalog number M100P). Such polyclonal antibody against full-length Her-2/neu would be expected to be able to bind to extracellular and cytoplasmic domains of Her-2/neu and not to be specific for the extracellular domain; such an antibody is still useful but it preferably should be combined with an antibody that is more specific towards Her-2/neu. Monoclonal antibodies are available against both the extracellular domain (e.g., R&D Systems Catalog number MAB1129) and the cytoplasmic domain (e.g., LABVISION neuAB-8) including against the C-terminal peptide (e.g., LABVISION neuAB-15). Monoclonal antibodies against Her-2/neu are also disclosed in Hudziak et al (1997, U.S. Pat. No. 5,677,171). One improved embodiment uses HERCEPTIN since binding with this antibody is best able to predict binding of HERCEPTIN as a treatment in the patient. Another advantageous embodiment uses a polyclonal antibody or a cocktail of antibodies binding to many epitopes on the Her-2/neu protein allows for higher sensitivity.

In one embodiment of this invention, one step of the immunoassay is performed on intact cancer cells by allowing one of the antibodies against Her-2/neu to bind to the cancer cells before the sample undergoes cell lysis. Such an antibody that is to be used to bind to intact cancer cells must be directed against the extracellular domain of Her-2/neu. Alternatively, the cancer cells can be lysed prior to all steps of the solution-based immunoassay and the immunoassay is performed on the cell lysate. In this case the immunoassay can utilize antibodies that bind selectively either to the extracellular or cytoplasmic domain of Her-2/neu. In a more specific embodiment of this invention the immunoassay uses one or two antibodies that bind selectively to the cytoplasmic domain of Her-2/neu.

In a preferable embodiment of the solution-based immunoassay of this invention, an antibody against the antigen is used to indirectly attach the antigen in solution to a solid support like a magnet, electrode or assay plate. An example of a solution-based immunoassay is an example using electrochemiluminescence (ECL) to detect antigen using two antibodies against the antigen in which one of the antibodies is labeled with ruthenium and the other is attached to a magnetic bead that can attach to an electrode. Besides electrochemiluminescence, other solution-based immunoassays that can yield a high sensitivity required for this application include, but are not limited to:
a) Chemiluminescence such as described by Liu Y et al., 2003 (J Food Protection 66:512-7).
b) Fluorogenic-chemiluminescence (FCL) as described by Yu H et al., 2000 (Biosens Bioelectron 14:829-40)
c) Fluorescence polarization immunoassay (see Howanitz J H, 1988 Arch Pathol Lab Med 112:775-9)
d) Time-resolved fluorescence immunoassay (Butcher H et al., 2003, J Immunol Methods 272:247-56; Soukka et al., 2001, Clin Chem 47:1269-78; Howanitz J H, 1988 Arch Pathol Lab Med 112:775-9)

In a preferable embodiment of the sandwich immunoassay of this invention, electrochemiluminescence (ECL) in which one of the antibodies is labeled with ruthenium and the other antibody is attached to a magnetic bead which can attach to an electrode is utilized.

Due to its sensitivity the method according to this invention for identifying patients likely to benefit from treatment with an anticancer agent that targets Her-2/neu can be fruitfully applied to patients from whom a tumor biopsy tissue had been previously determined (e.g. by immunohistochemistry or FISH analysis) to be negative for Her-2/neu expression by a tissue assay for Her-2/neu.

The invention will be better understood by reference to the following examples, which illustrate but do not limit the invention described herein.

### EXAMPLES

### Example 1

A patient with metastatic breast cancer comes into the office and a blood sample (8 to 40 mL) is withdrawn directly into BD Vacutainer CPT tubes containing an anticoagulant such as citrate. The material is centrifuged for 20 minutes at 1500 to 1800 RCF (relative centrifugal force). The cell layer above the gel barrier is removed and placed into a different container (e.g., tube) already containing an antibody to Her-2/neu such as a polyclonal antibody or HERCEPTIN (trastuzumab). Such an antibody has been previously labeled with ruthenium. Routine methods of ruthenium labeling the antibody are described in the art such as Lee et al., Am J Trop Med Hyg 2001, 65:1-9. The cells in the sample are then lyzed. Lysis can be achieved with any number of cell lysis reagents described in the art such as, but not limited to Lysis Buffer A [1% NP-40, 20 mM Tris (pH 8.0), 137 mM NaCl, 10% glycerol, 2 mM EDTA, 1 mM sodium orthovanadate, 10 ug/mL Aprotinin, 10 Ug/mL Leupeptin]. To the lysate, a second antibody which is biotinylated and which is against Her-2/neu is added. For example, this second antibody is a biotinylated polyclonal antibody against Her-2/neu such as from R&D systems (catalog number BAF1129). Next a solution of tripropylamine is added to the cell lysate along with streptavidin-coated magnetic beads that bring each of the two antibodies and the bound antigen close to an electrode. An electric current is applied and electrochemiluminescence (ECL) is detected using an ECL detection device such as one commercially available (BIOVERIS Corporation). Within these instruments is a photomultiplier tube (PMT) placed just above the working electrode for efficient light capture. Under the working electrode, a magnet is in place for capturing the beads coated with the target antigen. The signal is proportional to the amount of Her-2/neu found bound on the surface of the circulating tumor cells.

### Example 2

Methods identical to that used in example 1 are provided except that two polyclonal antibodies to Her-2/Neu are used for detection.

### Example 3

Methods are identical to that used in examples 1 and 2, except that the PBMC sample is lysed before each of the pair of antibodies are added.

### Example 4

Methods are identical to examples 1 and 3, except that a whole blood sample is directly used instead of the PBMC sample.

### Example 5

Methods identical to that used in examples 1 through 4 are provided except that the patient had a prior negative result for Her2/neu based on analysis of her primary tumor or does not have tumor tissue readily available for analysis.

### Example 6

A patient with a level of Her-2/neu above control samples as indicated in Examples 1-5 is deemed to have tumor cells positive for Her-2/neu and then treated with a regimen containing a monoclonal antibody against Her2/neu such as trastuzumab. A preferred treatment consists of trastuzumab at an initial loading dose of 4 mg/kg administered as a 90 minute infusion with a weekly maintenance dose of 2 mg/kg as a 30 minute infusion.

### Example 7

A patient with a level of Her-2/neu above control samples as indicated in Examples 1-5 is deemed to have tumor cells positive for Her-2/neu and then treated with a regimen containing lapatinib. An example of a dosing regimen with lapatinib is to give this agent as 1,2500 mg orally (five tablets of 250 mg each) once daily on days 1-21 in combination with capecitabine 2000 mg/m²/day (administered orally in 2 doses approximately 12 hours apart on Days 1-14 in a repeating 21 day cycle.

### Example 8

A patient with a level of Her-2/neu above control samples as indicated in Examples 1-5 is deemed to have tumor cells positive for Her-2/neu and then treated with a regimen containing CP-724,714. An example of a dosing regimen with CP-724,714 is to give this agent as oral doses of 250 mg twice daily.

### Example 9

A patient with a level of Her-2/neu above control samples as indicated in Examples 1-5 is deemed to have tumor cells positive for Her-2/neu and then treated with a regimen containing HKI-272. An example of a dosing regimen with HKI-272 is to give this agent as oral doses of 240 to 320 mg once on day 1 then once daily beginning on day 8.

### Example 10

In this example, purified recombinant Her-2/neu (extracellular domain) was used as a standard to examine the sensitivity of a solution-based immunoassay using electrochemiluminescence. This standard was diluted into PBS (phosphate buffered saline; pH=7.2) with 1% bovine serum albumin (BSA).

One assay buffer was prepared:
Assay Buffer 1: 0.5% Tween-20 and 0.5% bovine serum albumin (BSA) in PBS (phosphate buffered saline)

Her-2/neu standard (recombinant Her-2/neu extracellular domain) was obtained from Oncogene Science; Product #EL541). Goat anti-human Her-2/neu polyclonal antibody was obtained in both biotinylated and non-biotinylated forms (catalog numbers BAF1129 and AF1129, respectively) from R&D Systems, Inc. (Minneapolis, Minn. 55413 USA). The polyclonal antibody AF1129 was ruthenium labeled (“TAG-labeled”) using the procedures indicated in Lorence & Lu (PCT WO 2006/041959 A2).

The ruthenium-labeled polyclonal antibody AF1129 and the biotinylated polyclonal antibody BAF1129 are referred hereafter in this example and subsequent examples as “TAG-pAb” and “Biotin-pAb”.

An electrochemiluminecence assay was performed as follows:
Sequentially, Her-2/neu standards in 25 μl/well and then 50 μl/well of a mixture of TAG-Ab and Biotin-Ab [at one of two sets of concentrations (A) 1 μg/ml each in the 50 μl (a final concentration of 0.2 μg/ml in the final assay volume of 250 μl per well); and (B) at 0.7 μg/ml Biotin-pAb in the 50 μl (a final concentration of 0.14 μg/ml in the final assay volume of 250 μl per well); and 1.2 μg/ml TAG-pAb in the 50 μl (a final concentration of 0.24 μg/ml in the final assay volume of 250 μl per well)] diluted into the 4 PBS assay buffers) were added to wells of a 96-well U-bottom polypropylene plate and incubated at room temperature with constant shaking (e.g., for 2 hours).
10 μg of magnetic streptavidin beads (e.g., Dynabeads M-280 Streptavidin, BioVeris, Corporation, Gaithersburg, Md.) in 25 μl was added to each well and incubated with constant shaking (e.g., for 30 minutes).
Assay buffer 1 was added to each well to make a final volume of 250 μl per well. The amount of the analyte (recombinant Her-2/neu extracellular domain) in this assay was varied from 16 to 160 to 1600 pg per well. Control wells without analyte were also included. All conditions were tested in at least duplicate wells. The 96 well plate was then analyzed for electrochemiluminescence using the M-Series® 384 Analyzer (BioVeris, Corporation, Gaithersburg, Md.).

Results showed that all tested levels of recombinant Her-2/neu extracellular domain (16, 160 and 1600 pg/well) were detectable and above baseline using the solution-based immunoassay with TAG-pAb and Biotin-pAb at both sets of conditions used (Table 1).

TABLE 1
Electrochemiluminescence (ECL) detection of recombinant Her-2/neu
by immunoassay using ruthenium-labeled polyclonal (TAG-pAb)
and biotinylated polyclonal antibody (Biotin-pAb).

| | Mean ECL Signal (above background)* | |
|---|---|---|
| | Using final concentrations | Using final concentrations |
| Her-2/neu | of 0.2 μg/ml TAG-pAb and | of 0.24 μg/ml TAG-pAb and |
| (pg/well) | 0.2 μg/ml Biotin-pAb | 0.14 μg/ml Biotin-pAb |
| 16 | 85 | 54 |
| 160 | 473 | 510 |
| 1600 | 4404 | 6041 |

*Mean ECL signal above the mean signal from control wells with no antigen.

### Example 11

Methods as that used in example 10 were used except that: Cell extracts from SK-BR-3 breast carcinoma cells (positive control cells for Her-2/neu overexpression) and MDA-MB-468 breast carcinoma cells (negative for Her-2/neu overexpression) were analyzed.

SK-BR-3 and MDA-MB-468 cells (from ATCC, Manassas, Va.) were grown in 6-well tissue culture plates as per ATCC recommended conditions, washed two times with PBS, and an aliquot counted using a hemacytometer. Lysis of SK-BR-3 cells and obtaining the supernatant was performed using the Pierce Lysis Buffer (as described in in Lorence & Lu [PCT WO 2006/041959 A2]. The amount of lysate supernatant per well was varied from that extracted from 1 to 250 SK-BR-3 or MDA-MB-468 cells and analyzed for Her-2/neu using the immunoassay described in Experiment 10 using final assay concentrations of 0.2 μg/ml TAG-pAb and 0.2 μg/ml Biotin-pAb.

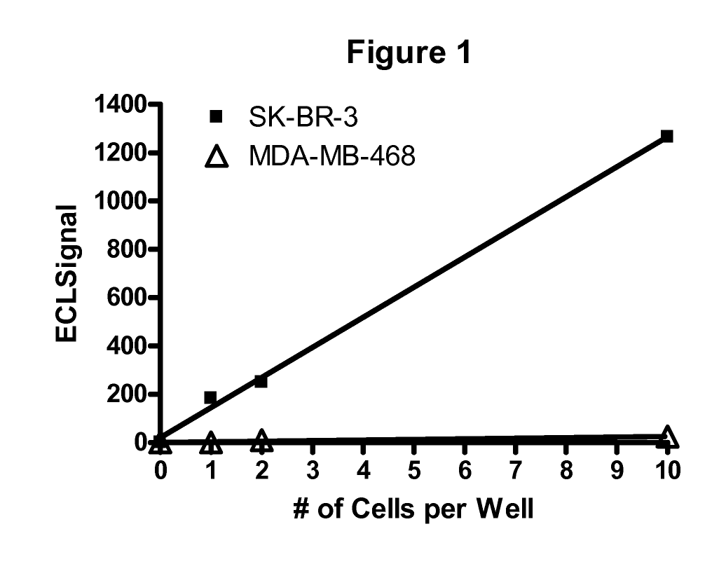

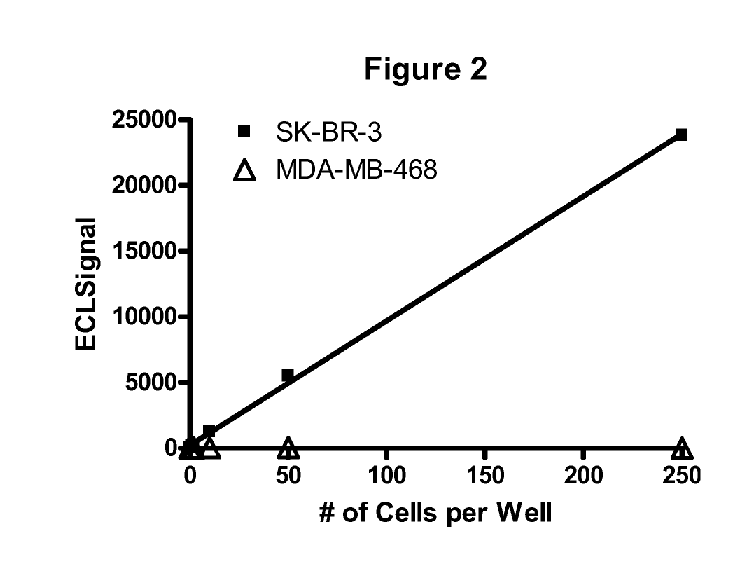

The results from this experiment are presented in FIGS. 1 and 2. FIG. 1 graphically displays the lower end of the data set to best see the ability of this assay to detect Her-2/neu from low cell numbers. FIG. 1 only includes data for the cell range up to 10 cells per well. FIG. 2 graphically displays the entire data set (up to 250 cells per well).

Her-2/neu was detectable and above baseline from lysates from SK-BR-3 cells in this experiment including those wells using the lowest amount of SK-BR-3 lysate in this experiment (lysate from 1 cells added per well; FIG. 1). Furthermore, the lysate from the SK-BR-3 cells (positive control for Her-2/neu overexpression) gave a much higher signal in the immunoassay for Her-2/neu than the lysate from MDA-MB-468 cells (negative for Her-2/neu overexpression) over the entire tested range from 1 to 250 cells per well, indicating the high specificity of the results for Her-2/neu detection (FIGS. 1 & 2).

### Example 12

In this experiment, immunoassay methods were as that used in example 10, except that human peripheral blood mononuclear cells (PBMCs) were obtained from a commercial source (Cellular Technology Ltd.; Cleveland, Ohio; product #CTL-UP1) and a lysate prepared in the same manner as for the breast cancer cells of example 11.

Samples for testing were prepared by adding the following into each well:
The cell lysate from 10 SK-BR-3 cells or control PBS assay buffer (PBS, pH=7.2 with 0.5% BSA and 0.5% Tween 20).
The cell lysate from 625,000 human PBMCs or control PBS assay buffer (PBS, pH=7.2 with 0.5% BSA and 0.5% Tween 20).

To these combined lysates, the following were added:
Tag-pAb and Biotin-pAb (each antibody at a final assay concentration of 0.2 μg/ml) was added per well and the 96-well plate was incubated with constant shaking for 2 hours at room temperature.
10 μg of magnetic streptavidin beads (e.g., Dynabeads M-280 Streptavidin, Catalog #110028, BioVeris, Corporation, Gaithersburg, Md.) in 25 μl was added to each well and incubated with constant shaking for 30 minutes.
PBS assay buffer (PBS, pH=7.2 with 0.5% BSA and 0.5% Tween 20) was added to each well to make a final volume of 250 μl per well. The 96 well plate was then analyzed for electrochemiluminescence as indicated in Example 10.

Results in this experiment are presented in Table 2. Her-2/neu was undetectable from 625,000 human PBMCs (Table 2). In contrast, Her-2/neu was detectable from even the smallest amount of SK-BR-3 breast cancer cell lysate used (lysate from 10 SK-BR-3 cell per well; see Table 6). Furthermore, the addition of cell lysates from 625,000 human PBMCs did not interfere with the detection of Her-2/neu in breast cancer cells (Table 2).

TABLE 2
ECL immunoassay detection of Her-2/neu in SK-BR-3
breast cancer cell lysates in the presence or
absence of lysates from human PBMCs.

| | |
|---|---|
| Lysates from | Mean ECL Signal (above background)* |

| SK-BR-3 Cells | | With lysates from |
|---|---|---|
| (SK-BR-3 | Without lysates | 625,000 PBMCs |
| cells/well) | from PBMCs | per well |
| 0 | 0 | Negative** |
| 10 | 608 | 388 |

*Background for these values is the signal from assay buffer only.
**Negative: ECL signal below background level.

### Example 13

In these two experiments, immunoassay were performed as in Example 10 using the Her-2/neu protein standard and in Example 11 using SK-BR-3 lysates, except the humanized monoclonal antibody trastuzumab (Herceptin) was used instead of Biotin-pAb and one of the following four ECL immunoassay conditions were tested:
Condition #1: Trastuzumab was directly labeled with ruthenium. Biotinylated polyclonal antibody at a final concentration assay concentration of 0.2 μg/ml as indicated in Example 10 was used.
Condition #2: Trastuzumab was directly labeled with biotin. Ruthenium-labeled polyclonal antibody at a final assay concentration of 0.2 μg/ml as indicated in Example 10 was used.
Conditions #3 and 4: In these two conditions, trastuzumab was not directly labeled; instead a secondary antibody (biotin-labeled anti-human IgG) was used instead.
   Condition #3: Trastuzumab (final concentration of 0.2 μg/ml when ECL is performed in 250 μl per well) was first incubated directly with the cell lysates for 50 minutes. This step was then followed by addition of Biotin-labeled anti-human IgG (final concentration of 0.2 μg/ml; eBioscience, San Diego, Calif.; catalog #13-4998, biotin-labeled goat antibody against human IgG) and TAG-pAb (final concentration of 0.2 μg/ml) and then these assay constituents were incubated for 1 hour at room temperature followed by the addition of streptavidin-beads (as used previously in Example 10).
   Condition #4: Concentrations were as in condition #3, except that the order of the reagent additions was changed. In condition #4, trastuzumab plus ruthenium-labeled pAb (TAG-pAb) were first incubated with cell lysates for 50 minutes. This step was then followed by the addition of biotin-labeled anti human IgG and then these assay constituents were incubated for 1 hour at room temperature followed by the addition of streptavidin-beads (as used previously in Example 10).

In the first experiment using trastuzumab, conditions #1 and #2 were tested in assaying for Her-2/neu positive control samples. In this experiment, condition #1 yielded a signal well above background using 1600 pg/well of Her-2/neu and was more sensitive than condition #2.

TABLE 3A
ECL immunoassay detection of Her-2/neu standards in SK-BR-3
breast cancer cell lysates using labeled trastuzumab according
to conditions #1 and #2 (see text in this Example
13 for details of the two conditions.

| | |
|---|---|
| Her-2/neu | Mean ECL Signal (above background) |

| (pg/well) | Condition 1 | Condition 2 |
|---|---|---|
| 1 | Negative* | Negative* |
| 4 | 14 | 2 |
| 16 | 11 | 1 |
| 160 | 21 | Negative* |
| 1600 | 81 | 14 |

*Negative: ECL signal below background level.

In the next experiment using trastuzumab, conditions #3 and #4 were devised to improve sensitivity relative to conditions #1 and #2 and were used to assay Her-2/neu from lysates of SK-Br-3 cells. The results showed that under conditions #3 and #4 that trastuzumab can be used together with a polyclonal antibody in a solution-based assay to detect Her-2/neu from lysates from at least 1 SK-BR-3 cell per well (Table 3B). Condition #4 was more sensitive than condition #3 at detecting low numbers of SK-BR-3 cells.

TABLE 3B
ECL immunoassay detection of Her-2/neu in SK-BR-3
breast cancer cell lysates using unlabeled trastuzumab,
Biotin-labeled anti-human IgG, and TAG-pAb.

| Lysates from | |
|---|---|
| SK-BR-3 Cells | |
| (SK-BR-3 | Mean ECL Signal (above background) |

| cells/well) | Condition 3 | Condition 4 |
|---|---|---|
| 0 | 0 | 0 |
| 1 | 156 | 290 |
| 2 | 213 | 311 |
| 10 | 841 | 758 |
| 50 | 3455 | 3481 |
| 250 | 15065 | 15147 |

Two different immunoassays conditions (condition #3 and condition #4) were tested as defined in the text for this Example 13.

Based on the results of this example, among the four conditions tested using trastuzumab, conditions #3 and #4 which used a secondary antibody against trastuzumab gave advantageous results relative to conditions #1 and #2 in which trastuzumab was directly labeled and no secondary antibody was used.

### Example 14

In this experiment, immunoassay methods using TAG-pAb and Biotin-pAb were as that used in example 12 and immunoassay methods using trastuzumab were identical as in condition #4 of Example 13. Human peripheral blood mononuclear cells (PBMCs) from six additional donors were obtained from Cellular Technology Ltd.; Cleveland, Ohio; product #CTL-UP1) and lysates prepared as in Example 12. In this example, lysates from PBMCs were tested at 500,000 to 1,000,000 cells per well either with or without the lysate from SK-BR-3 cells (tested at 10 cells per well) spiked into these PBMC lysates.

Results using TAG-pAb and Biotin-pAb are shown in Tables 4-9 for individual PBMC donors and in Table 10 for the mean from all donors. As shown in Tables 4-9 and summarized in Table 10, very consistent results across all donor PBMCs were obtained. Lysates from even as high as 1,000,000 PMBCs all gave markedly lower signals for Her-2/neu than lysates from just 10 SK-BR-3 cells. A high signal from the lysates from 10 SK-BR-3 cells could be obtained even in the presence of lysates from as high as 1,000,000 PBMCs. These results indicate that human PBMCs did not interfere with the ability of this immunoassay to detect Her-2/neu from small numbers of breast cancer cells overexpressing Her-2/neu.

Results using trastuzumab, biotin-labeled anti-human IgG and TAG-pAb are shown in Tables 11-16 for individual PBMC donors and in Table 17 for the mean from all donors. Very similar results were obtained using a solution-based immunoassay with these antibodies as were obtained when using polyclonal antibody for both TAG-label and Biotin-label. Again, the results were consistent across all donor PBMCs used. Lysates from 1,000,000 PMBCs from every donor gave no signal above baseline for Her-2/neu in contrast to the high signal obtained from lysates from just 10 SK-BR-3 cells. These results again indicate that human PBMCs did not interfere with the ability of this approach to detect Her-2/neu from small numbers of breast cancer cells overexpressing Her-2/neu.

TABLE 4
ECL immunoassay detection (using Biotin-pAb and Tag-pAb) of
Her-2/neu in SK-BR-3 breast cancer cell lysates in the presence
or absence of lysates from human PBMCs from donor #0706.

| | |
|---|---|
| Lysates from | Mean ECL Signal (above background)* |

| SK-BR-3 Cells | | With lysates from |
|---|---|---|
| (SK-BR-3 | Without lysates | 1,000,000 PBMCs |
| cells/well) | from PBMCs | per well |
| 0 | 0 | 177 |
| 10 | 789 | 1174 |

*Background for these values is the signal from assay buffer only.

TABLE 5
ECL immunoassay detection (using Biotin-pAb and Tag-pAb) of
Her-2/neu in SK-BR-3 breast cancer cell lysates in the presence
or absence of lysates from human PBMCs from donor #0920.

| Lysates from | Mean ECL Signal (above background)* | | |
|---|---|---|---|
| SK-BR-3 Cells | | With lysates from | With lysates from |
| (SK-BR-3 | Without lysates | 500,000 PBMCs | 1,000,000 PBMCs |
| cells/well) | from PBMCs | per well | per well |
| 0 | 0 | 77 | 87 |
| 10 | 789 | 1008 | 800 |

*Background for these values is the signal from assay buffer only.

TABLE 6
ECL immunoassay detection (using Biotin-pAb and Tag-pAb) of
Her-2/neu in SK-BR-3 breast cancer cell lysates in the presence
or absence of lysates from human PBMCs from donor #1026.

| Lysates from | Mean ECL Signal (above background)* | | |
|---|---|---|---|
| SK-BR-3 Cells | | With lysates from | With lysates from |
| (SK-BR-3 | Without lysates | 500,000 PBMCs | 1,000,000 PBMCs |
| cells/well) | from PBMCs | per well | per well |
| 0 | 0 | 162 | 137 |
| 10 | 789 | 895 | 865 |

*Background for these values is the signal from assay buffer only.

TABLE 7
ECL immunoassay detection (using Biotin-pAb and Tag-pAb) of
Her-2/neu in SK-BR-3 breast cancer cell lysates in the presence
or absence of lysates from human PBMCs from donor #0711.

| Lysates from | Mean ECL Signal (above background)* | | |
|---|---|---|---|
| SK-BR-3 Cells | | With lysates from | With lysates from |
| (SK-BR-3 | Without lysates | 500,000 PBMCs | 1,000,000 PBMCs |
| cells/well) | from PBMCs | per well | per well |
| 0 | 0 | 167 | 75 |
| 10 | 789 | 805 | 1052 |

*Background for these values is the signal from assay buffer only.

TABLE 8
ECL immunoassay detection (using Biotin-pAb and Tag-pAb) of
Her-2/neu in SK-BR-3 breast cancer cell lysates in the presence
or absence of lysates from human PBMCs from donor #0524.

| Lysates from | Mean ECL Signal (above background)* | | |
|---|---|---|---|
| SK-BR-3 Cells | | With lysates from | With lysates from |
| (SK-BR-3 | Without lysates | 500,000 PBMCs | 1,000,000 PBMCs |
| cells/well) | from PBMCs | per well | per well |
| 0 | 0 | 210 | 340 |
| 10 | 789 | 683 | 1180 |

*Background for these values is the signal from assay buffer only.

TABLE 9
ECL immunoassay detection (using Biotin-pAb and Tag-pAb) of
Her-2/neu in SK-BR-3 breast cancer cell lysates in the presence
or absence of lysates from human PBMCs from donor #0116G.

| Lysates from | Mean ECL Signal (above background)* | | |
|---|---|---|---|
| SK-BR-3 Cells | | With lysates from | With lysates from |
| (SK-BR-3 | Without lysates | 500,000 PBMCs | 1,000,000 PBMCs |
| cells/well) | from PBMCs | per well | per well |
| 0 | 0 | 121 | 86 |
| 10 | 789 | 618 | 1137 |

*Background for these values is the signal from assay buffer only.

TABLE 10
Average of data from Tables 4-9: ECL immunoassay
detection (using Biotin-pAb and Tag-pAb) of Her-2/neu
in SK-BR-3 breast cancer cell lysates in the presence
or absence of lysates from human PBMCs.

| Lysates from | Mean ECL Signal (above background)* | | |
|---|---|---|---|
| SK-BR-3 Cells | | With lysates from | With lysates from |
| (SK-BR-3 | Without lysates | 500,000 PBMCs | 1,000,000 PBMCs |
| cells/well) | from PBMCs | per well | per well |
| 0 | 0 | 147 | 150 |
| 10 | 789 | 678 | 1035 |

Shown are the mean values using the data from all donor PBMCs.
*Background for these values is the signal from assay buffer only.

TABLE 11
ECL immunoassay detection (using trastuzumab, Biotin-labeled
anti-human IgG, and Tag-pAb) of Her-2/neu in SK-BR-3 breast
cancer cell lysates in the presence or absence of lysates
from human PBMCs from donor #0706.

| | |
|---|---|
| Lysates from | Mean ECL Signal (above background)* |

| SK-BR-3 Cells | | With lysates from |
|---|---|---|
| (SK-BR-3 | Without lysates | 1,000,000 PBMCs |
| cells/well) | from PBMCs | per well |
| 0 | 0 | Negative** |
| 10 | 428 | 314 |

*Background for these values is the signal from assay buffer only.
**Negative: ECL signal slightly below background level.

TABLE 12
ECL immunoassay detection (using trastuzumab, Biotin-labeled
anti-human IgG, and Tag-pAb) of Her-2/neu in SK-BR-3 breast
cancer cell lysates in the presence or absence of lysates
from human PBMCs from donor #0920.

| Lysates from | Mean ECL Signal (above background)* | | |
|---|---|---|---|
| SK-BR-3 Cells | | With lysates from | With lysates from |
| (SK-BR-3 | Without lysates | 500,000 PBMCs | 1,000,000 PBMCs |
| cells/well) | from PBMCs | per well | per well |
| 0 | 0 | Negative** | Negative** |
| 10 | 428 | 492 | 269 |

*Background for these values is the signal from assay buffer only.
**Negative: ECL signal slightly below background level.

TABLE 13
ECL immunoassay detection (using trastuzumab, Biotin-labeled
anti-human IgG, and Tag-pAb) of Her-2/neu in SK-BR-3 breast
cancer cell lysates in the presence or absence of lysates
from human PBMCs from donor #1026.

| Lysates from | Mean ECL Signal (above background)* | | |
|---|---|---|---|
| SK-BR-3 Cells | | With lysates from | With lysates from |
| (SK-BR-3 | Without lysates | 500,000 PBMCs | 1,000,000 PBMCs |
| cells/well) | from PBMCs | per well | per well |
| 0 | 0 | Negative** | Negative** |
| 10 | 428 | 452 | 311 |

*Background for these values is the signal from assay buffer only.
**Negative: ECL signal slightly below background level.

TABLE 14
ECL immunoassay detection (using trastuzumab, Biotin-labeled
anti-human IgG, and Tag-pAb) of Her-2/neu in SK-BR-3 breast
cancer cell lysates in the presence or absence of lysates
from human PBMCs from donor #0711.

| Lysates from | Mean ECL Signal (above background)* | | |
|---|---|---|---|
| SK-BR-3 Cells | | With lysates from | With lysates from |
| (SK-BR-3 | Without lysates | 500,000 PBMCs | 1,000,000 PBMCs |
| cells/well) | from PBMCs | per well | per well |
| 0 | 0 | Negative** | Negative** |
| 10 | 428 | 397 | 525 |

*Background for these values is the signal from assay buffer only.
**Negative: ECL signal slightly below background level.

TABLE 15
ECL immunoassay detection (using trastuzumab, Biotin-labeled
anti-human IgG, and Tag-pAb) of Her-2/neu in SK-BR-3 breast
cancer cell lysates in the presence or absence of lysates
from human PBMCs from donor #0524.

| Lysates from | Mean ECL Signal (above background)* | | |
|---|---|---|---|
| SK-BR-3 Cells | | With lysates from | With lysates from |
| (SK-BR-3 | Without lysates | 500,000 PBMCs | 1,000,000 PBMCs |
| cells/well) | from PBMCs | per well | per well |
| 0 | 0 | 79 | Negative** |
| 10 | 428 | 468 | 496 |

*Background for these values is the signal from assay buffer only.
**Negative: ECL signal slightly below background level.

TABLE 16
ECL immunoassay detection (using trastuzumab, Biotin-labeled
anti-human IgG, and Tag-pAb) of Her-2/neu in SK-BR-3 breast
cancer cell lysates in the presence or absence of lysates
from human PBMCs from donor #0116G.

| Lysates from | Mean ECL Signal (above background)* | | |
|---|---|---|---|
| SK-BR-3 Cells | | With lysates from | With lysates from |
| (SK-BR-3 | Without lysates | 500,000 PBMCs | 1,000,000 PBMCs |
| cells/well) | from PBMCs | per well | per well |
| 0 | 0 | Negative** | Negative** |
| 10 | 428 | 217 | 545 |

*Background for these values is the signal from assay buffer only.
**Negative: ECL signal slightly below background level.

TABLE 17
Average of data from Tables 11-16: ECL immunoassay detection
(using trastuzumab, Biotin-labeled anti-human IgG, and Tag-pAb)
of Her-2/neu in SK-BR-3 breast cancer cell lysates in the
presence or absence of lysates from human PBMCs.

| Lysates from | Mean ECL Signal (above background)* | | |
|---|---|---|---|
| SK-BR-3 Cells | | With lysates from | With lysates from |
| (SK-BR-3 | Without lysates | 500,000 PBMCs | 1,000,000 PBMCs |
| cells/well) | from PBMCs | per well | per well |
| 0 | 0 | Negative** | Negative** |
| 10 | 428 | 362 | 410 |

Shown are the mean values using the data from all donor PBMCs.
*Background for these values is the signal from assay buffer only.
**Negative: ECL signal slightly below background level.

1. A method of detecting the expression of Her-2/neu protein on circulating cancer cells in a whole blood sample, comprising performing on the blood sample an immunoassay capable of detecting cancer cell-associated Her-2/neu, in which a positive immunoassay result indicates the presence of Her-2/neu on the cancer cells;
wherein the circulating cancer cells are not isolated from the whole blood prior to the performance of the immunoassay;
and wherein the immunoassay:
a) is capable of detecting Her-2/neu from SK-BR-3 breast cancer cells when spiked into blood at a concentration of less than or equal to 100 SK-
b) BR-3 cells per milliliter of blood; and
is capable of detecting Her-2/neu from 10 SK-BR-3 breast cancer cells when assayed in the presence of at least 1 million human peripheral blood mononuclear cells.

2. A method of detecting the expression of Her-2/neu protein on circulating cancer cells in a blood sample, comprising performing on the blood sample an immunoassay capable of detecting cancer cell-associated Her-2/neu, in which a positive immunoassay result indicates the presence of Her-2/neu on the cancer cells;
wherein the circulating cancer cells are not isolated from peripheral blood mononuclear cells prior to the performance of the immunoassay;
and wherein the immunoassay:
a) is capable of detecting Her-2/neu from SK-BR-3 breast cancer cells when spiked into blood at a concentration of less than or equal to 100 SK-BR-3 cells per milliliter of blood; and
b) is capable of detecting Her-2/neu from 10 SK-BR-3 breast cancer cells when assayed in the presence of at least 1 million human peripheral blood mononuclear cells.

3. The method of claim 1, wherein the immunoassay is a solution-based immunoassay, and said immunoassay uses a technique selected from the group consisting of electrochemiluminescence, chemiluminescence, fluorogenic chemiluminescence, fluorescence polarization, and time-resolved fluorescence, for detection.

4. (canceled)

5. The method of claim 1, wherein the immunoassay is a sandwich immunoassay and said immunoassay uses a technique selected from the group consisting of electrochemiluminescence, chemiluminescence, and fluorogenic chemiluminescence, for detection.

6. (canceled)

7. The method of claim 1, wherein the immunoassay generates a signal proportional to the number of cancer cell-associated Her-2/neu molecules present in the blood sample.

8-11. (canceled)

12. The method of claim 1, wherein the immunoassay uses a monoclonal antibody against Her-2/neu.

13. The method of claim 12, wherein the immunoassay uses a secondary antibody against said monoclonal antibody.

14. The method of claim 12, wherein the monoclonal antibody is a humanized mouse monoclonal antibody.

15. The method of claim 14, wherein the monoclonal antibody is trastuzumab.

16-25. (canceled)

26. The method of claim 2, wherein the immunoassay is a solution-based immunoassay, and said immunoassay uses a technique selected from the group consisting of electrochemiluminescence, chemiluminescence, fluorogenic chemiluminescence, fluorescence polarization, and time-resolved fluorescence, for detection.

27. The method of claim 2, wherein the immunoassay is a sandwich immunoassay and said immunoassay uses a technique selected from the group consisting of electrochemiluminescence, chemiluminescence, and fluorogenic chemiluminescence, for detection.

28. The method of claim 2, wherein the immunoassay generates a signal proportional to the number of cancer cell-associated Her-2/neu molecules present in the blood sample.

29. The method of claim 2, wherein the immunoassay uses a monoclonal antibody against Her-2/neu.

30. The method of claim 29, wherein the immunoassay uses a secondary antibody against said monoclonal antibody.

31. The method of claim 29, wherein the monoclonal antibody is a humanized mouse monoclonal antibody.

32. The method of claim 31, wherein the monoclonal antibody is trastuzumab.
